(19) United States
(12) Patent Application Publication
(10) Pub. No.: US 2025/0375481 A1
(43) Pub. Date: Dec. 11, 2025

(54) METHODS AND PROCESSES FOR MANUFACTURE OF A TOPICALLY ADHERENT SELECTIVE BACTERICIDE

(71) Applicant: LINTBELLS LIMITED, Weston, Hitchin Hertfordshire (GB)

(72) Inventors: Glenn Francis Vile, Weston, Hitchin Hertfordshire (GB); Ann Kimberly Haigh, Weston, Hitchin Hertfordshire (GB)

(73) Assignee: LINTBELLS LIMITED, Weston, Hitchin Hertfordshire (GB)

(21) Appl. No.: 18/865,540

(22) PCT Filed: May 12, 2023
(86) PCT No.: PCT/GB2023/051257

§ 371 Date: Nov. 13, 2024

(30) Foreign Application Priority Data
May 13, 2022 (GB) 2207004.9

(51) Int. Cl. A61K 35/20; A61K 9/06; A61K 38/01; A61P 31/04

(52) U.S. Cl. CPC A61K 35/20; A61K 9/06; A61K 38/018; A61P 31/04

(57) ABSTRACT

Disclosed herein are processes for the manufacture of a new topically adherent selective bactericide and products resulting therefrom. More specifically a method for producing a complexed stable microbiome regulating protein configured for topical application and use of that product as a medicament, effective for therapeutic effect against pathogenic bacteria, but not commensal bacteria.

### RELATED APPLICATIONS

This application is a national phase application filed under 35 USC § 371 of PCT Application No. PCT/GB2023/051257 with an International filing date of May 12, 2023, which claims priority of GB Patent Application 2207004.9 filed May 13, 2022. Each of these applications is herein incorporated by reference in its entirety for all purposes.

### BACKGROUND OF THE INVENTION

Bacteria readily attach to surfaces and create biofilms. Biofilms are commonly found on all epithelial surfaces in humans and animals including the skin, gut, lungs, nasal and oral cavities. The bacteria contained within any biofilm can be benign or even beneficial (commensal) such as bacteria from the Lactobacillus family, or pathogenic, adverse to the host causing disease and illness e.g. E. coli and S. aureus bacteria. In most cases, the biofilm is beneficial or at least benign because the predominant components of the biofilm are commensal bacteria. However, the effect of the biofilm can become adverse when the level of pathogenic bacteria in the film increases and overwhelms the benign or commensal bacteria. For example several skin diseases are a result of over proliferation of pathogenic bacteria on the skin, e.g. atopic dermatitis is a consequence of over population by the pathogenic bacteria S. aureus on the skin. In particular, decay and/or gum and oral disease may result if pathogenic bacteria become predominant in the biofilm (plaque) covering the gum and teeth in the oral cavity of animals. For example, periodontal disease is one of the most commonly diagnosed oral diseases in dogs and cats often due to adverse changes in the plaque.

Dental prophylaxis has a major impact on the oral and plaque microbiotas, although prevention and treatment concerning plaque-associated diseases may require more specificity targeted therapy because general oral samples are a poor proxy of the plaque bacteria (and those responsible in periodontal disease). Although not solely responsible, psychrobacter are an example of a genus likely to be highly relevant given its specific predominance in the plaque of dogs. Flancman R, Singh A, Weese J S (2018) Evaluation of the impact of dental prophylaxis on the oral microbiota of dogs. PLOS ONE 13(6): e0199676.

https://doi.org/10.1371/journal.pone.0199676. Further, another study has linked psychrobacter as being the bacteria responsible for general mal-odour in dogs; Meason-Smith et al. Vet Dermatol 2018; 29:465-e158. Physical dental cleaning of the plaque significantly reduces psychrobacter levels but the effect is temporary; after cleaning, a reversion to baseline levels is observed within five weeks. Therefore, a regular and specific therapy targeting the plaque is required to facilitate effective treatment and/or maintenance of oral health in dogs, as compared to general oral therapy.

Applicants in the art have previously attempted to solve this problem by providing a malleable product with a flexible matrix, which provides a mechanical cleansing action to remove build-up in bacteria, such as U.S. Pat. No. 5,407,661. However, this does not provide a selective removal of pathogenic bacteria. To prevent and avoid disease/illness, compositions for modifying biofilms often include a bactericidal agent and other component in combination, e.g. phospholipid, surfactant or enzyme. These compositions bind well to epithelial surfaces to destroy pathogens effectively but since the active agent is non-selective, it acts to destroy the natural biofilm including the beneficial commensal bacteria. Standard veterinary oral care products kill all the bacteria present in the mouth of a canine.

A number of strategies have been developed to modify biofilms on epithelial surfaces with the aim of disrupting or removing the biofilm all together, and/or decreasing the levels of pathogenic bacteria in the biofilm, usually by the administration of a bactericidal agent.

For example, NZ763741 describes the use of a phospholipid to disrupt the biofilm and an antibiotic to non-selectively kill the bacteria in the biofilm.

NZ779091 describes the use of a surfactant and a bactericidal agent to disperse the biofilm and non-selectively kill the bacteria constituting the biofilm.

NZ757876 describes the use of the non-selective bactericidal agent triacyl polyamine to kill and disperse the bacteria in a biofilm.

NZ732061 describes the use of applying oxidoreductase enzymes and substrates for these enzymes such as honey to kill pathogenic bacteria in chronic wounds and medical devices. The bactericidal agent produced by oxidoreductase enzymes is the non-selective bactericidal agent hydrogen peroxide.

NZ755166 describes the use of a thiol based antioxidant, an enzyme to breakdown the extracellular matrix, and a non-selective bactericidal agent such as an antibiotic or antiseptic.

In each publication noted above, the strategies involve:
The use of components that disrupt the naturally occurring biofilm; and/or
The use of bactericidal components that do not discriminate between helpful commensal bacteria and harmful pathogenic bacteria in the naturally occurring biofilm.

While most bactericidal agents are generally non-selective, recently agents have been described that selectively kill pathogenic bacteria while not effecting the growth of commensal bacteria. For example PCT/NZ2017/050043 describe that a combination of protein fractions from bovine dairy milk (IDP™) can be effective at preventing the growth of pathogenic bacteria such as E. coli and S. aureus while not effecting the growth of commensal bacteria from, for example, the Lactobacillus family. However, there is no evidence that IDP™ binds to epithelial surfaces. IDP™ or ‘Immune Defence Protein’ is a microbiome-regulating fraction of proteins from cows' milk. This fraction is described in at least US12/304108, NZ719276, NZ742157, PCT/NZ2017/050043 and NZ744458. According to published literature, IDP™ is a formulation based on milk bioactive proteins that is extracted from milk. The components of IDP™ are produced naturally by the cow as an immune defence response against infection and inflammation. IDP™ is reported to have anti-inflammatory, antioxidant and antimicrobial action in vitro that selectively supports ‘good’ bacteria flora and kills ‘bad’ bacteria. IDP™ is already used for oral, throat, gut and skin applications.

Despite potential advantage, it has been shown that said proteins do not alone enable effective use of their properties and formulations encompassing these agents or similar types of functioning agent appear not to successfully deliver the required effect at the intended location or provide that activity over extended periods.

Fibraspect™ is a known protein-based gel which may be used as an alternative to surfactant emulsifying agents. The gel has the ability to bind active ingredients together, release the agents in a controlled manner and has shear thinning rheology (thixotropic) giving it good stability and positive skin feel. It has a semi-solid material composed of soluble native protein, protein aggregates and protein fibrils produced from whey protein isolates (WPIs) to form a modified WPI. The modified WPI gels have a viscosity from 0.25 to 4.5 Poise.

The problem of providing a suitable oral product, which is inherently effective to deliver and enable a specific effect in the oral mucosa of animals is largely unmet to date. In particular, adequately targeting plaque without harming the commensal flora is a challenge. Thus, effective solutions which are both suitable for and successful in maintaining or improving oral health of companion animals are extremely limited to date.

### SUMMARY OF INVENTION

A new protein complex, methods of making the same and uses and compositions comprising that protein complex are defined herein.

Firstly, this invention concerns a bio-complex that comprises a unique combination of whey protein isolate; a semi-solid material composed of soluble native protein, protein aggregates and protein fibrils produced from whey protein isolates (WPIs) to form a modified WPI. Adherence of an bio-agent which retains selective antibacterial activity at an epithelial surface enables the functionality of useful selective agents to be applied in biological environments that have previously not been possible. The bio-complex is a complexed protein formed by combining the microbiome regulating proteins with the modified WPI by a new and innovative process.

The new stable bio-complex invention has enabled the functionality of selective agents to potentially useful in biological environments. Specifically, adherence of an agent which retains selective antibacterial activity at an epithelial surface, is a further step toward a useful solution.

The new bio-complex is formed by combining the microbiome regulating proteins with the modified WPI in a new and unique way. This enables selective bactericidal activity to remain while forming the epithelial-adherent characteristic in the composition. In-vitro testing has confirmed these properties remain within the resulting powder

Furthermore, the formulation of the bio-complex maybe more crucial to enabling biological effect in some applications, particularly veterinary applications where the subject cannot take instruction. For example, effective use of an antibacterial is possible, in part, by comprehension and execution of an instruction, e.g. rubbing a surface, washing/swilling within a cavity, rather than swallowing. However, by contrast for effective veterinary use (e.g. a pet dog or cat) the formulation itself must inherently deliver and enable the biological activity, regardless of associated instruction.

Thus, a new capability to adhere a specific agent to an epithelial surface of an animal, such as a mucosal surface, as well as useful formulation thereof directed to that purpose will be very helpful for the veterinary field particularly.

Successful delivery of a selective agent in animals, including dogs and cats and by a suitable formulated means (which inherently permits effective use of the active) is therefore a very useful advantage.

Described herein are the methods and a complexed protein with useful effects in terms of stabilisation and, when applied topically, the ability to adhere the complexed protein to an epithelial surface and retain protein functionality.

There is provided a method of producing a complexed stable microbiome regulating protein, bio-complex, configured for topical application, the method comprising the steps of:
selecting a microbiome regulating protein in a powder form;
selecting a modified whey protein isolate (WPI) from milk in a gel form;
solubilising the microbiome regulating protein in an aqueous salt solution, the aqueous salt solution having an ionic strength of 25-200 mM NaCl;
blending together the solubilised microbiome regulating protein and modified WPI;
adjusting the pH to 2.0-6.0;
optionally adjusting the temperature to 18-37° C.;

holding the blend at a pH of 2.0-6.0 and a temperature of 18-37° C. for at least 30 minutes.

The invention further comprises a complexed protein produced by the method substantially as described above.

This process enables production of a new biocomplex composition in which selective bactericidal activity remains, whilst benefiting from the epithelial-adherent characteristics. In-vitro testing of the biocomplex composition has confirmed these advantageous properties remain within the resulting powder.

The invention also comprises a complexed protein comprising microbiome functional protein complexed with a modified whey protein isolate (WPI) from milk, the complexed microbiome functional protein configured for topical application and, on application, to adhere to epithelial surfaces.

The invention further concerns a method of selectively treating an animal for a toxic pathogenic bacteria, but minimising any reduction in commensal bacteria population, by the step of topically administering a complexed protein, the complexed protein comprising a microbiome functional protein complexed with a modified whey protein isolate (WPI) from milk, the complexed protein configured for topical application and, on application, configured to adhere to epithelial surfaces.

Further, there is provided the use of a complexed protein comprising a microbiome functional protein complexed with a modified whey protein isolate (WPI) from milk, the complexed protein configured for topical application and, on application configured to adhere to epithelial surfaces, in the manufacture of a medicament for topical treatment of toxic pathogenic bacteria, but minimising any reduction in commensal bacteria populations on epithelial surfaces of an animal. The inventor has identified a complexed protein with useful effects in terms of stabilisation and, when applied topically, the ability to adhere the functional proteins described to an epithelial surface and retain excellent protein/protein fraction functionality. This appears to be at least in part due to the important chemical and physical conditions described further below under which the two components are reacted together to form the complexed protein which may be controlled to optimise the functional activity of both components. Through careful balance of these parameters, the resulting useful effects and the extent of adherence of protein on epithelial surfaces post complexing was more than double that of each component alone and, in the inventors experience, synergistic in adherence and functionality, or at least well beyond that anticipated.

The invention extends to the complexed protein or biocomplex as previously described for use as a medicament, optionally for use as a veterinary medicament.

In particular, that use may be for the treatment of an oral or dental infection, optionally gum or periodontal disease, mediated by an imbalance of pathogenic bacteria. In embodiments, the composition comprises the complexed protein or biocomplex described herein above in a therapeutically effective amount.

Further re-formulation of the bio-complex maybe more crucial to enabling biological effect in some applications. As such the invention extends to a composition comprising: a bio-complex comprising solubilised microbiome regulating protein(s) complexed with a modified whey protein isolate (WPI) from milk, wherein said biocomplex adheres to epithelial surfaces and is selectively toxic to pathogenic bacteria, but not to commensal bacteria. The bio-complex may include solubilised microbiome regulating proteins complexed with a modified whey protein isolate (WPI) from milk. In embodiments, one or more of microbiome regulating proteins of the biocomplex are selected from lactoperoxidase, lactoferrin, lysomal alpha-mannosidase, immunoglobulin G, angiogenin, ribonuclease 4, and quiescin sulfhydryl oxidase.

The therapeutic effect may be further improved by ensuring delivery of the bio-complex to a precise location e.g. gums of a dog. As the biocomplex is situated in close proximity to the teeth and gum line it adheres effectively on to epithelial surface in the animal's mouth.

Further aspects and advantages of the methods and complex and uses described will become apparent from the ensuing description that is given by way of example only.

### BRIEF DESCRIPTION

Further aspects of the methods and complexed protein described will become apparent from the following description that is given by way of example only and with reference to the accompanying drawings in which:

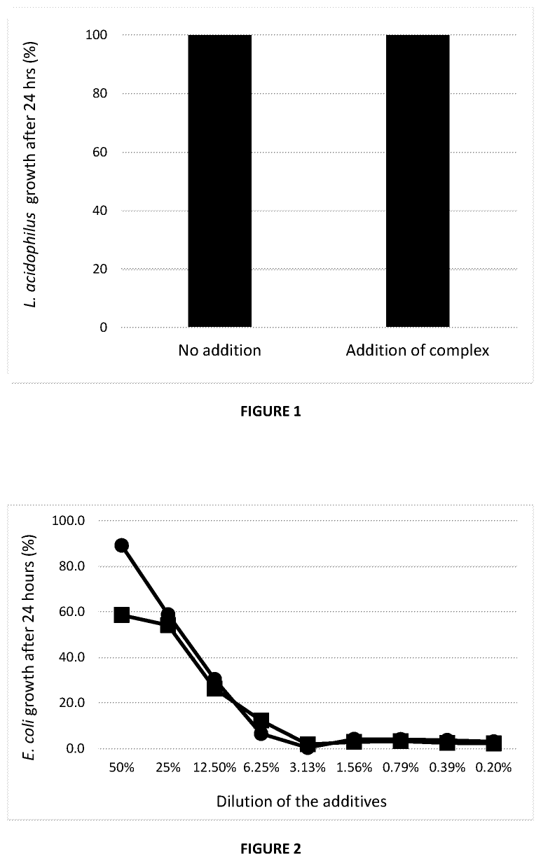

FIG. 1 is a graph showing the nil effect of the complex on the growth of the commensal bacteria L. acidophilus;

FIG. 2 is a graph showing the bactericidal effect of the complex to the pathogenic bacteria E. coli;

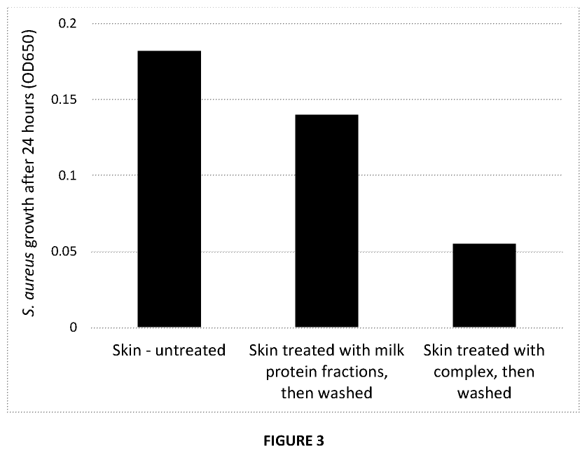

FIG. 3 is a graph showing that the complex adheres well to epithelial surfaces and retains its bactericidal activity towards the pathogenic bacteria S. aureus;

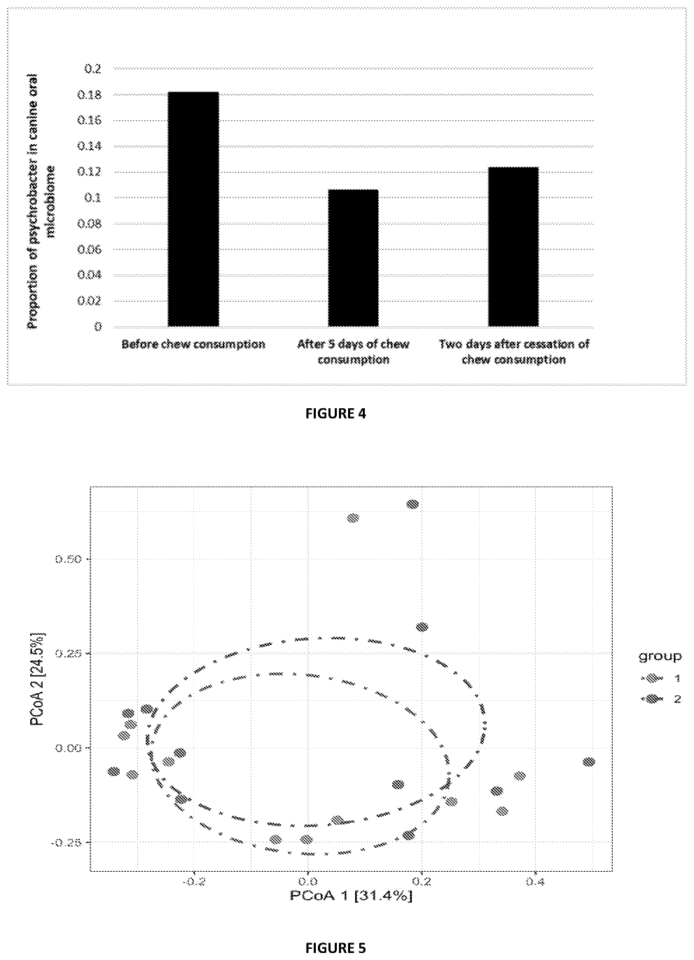

FIG. 4 is a graph showing the effect on the oral pathogen psychrobacter after use of the protein biocomplex in canaines, according to an embodiment of the invention for 5 days;

FIG. 5 is a graph showing the effect on canine gut microbiome, after use of the protein biocomplex in according to an embodiment of the invention for 5 days;

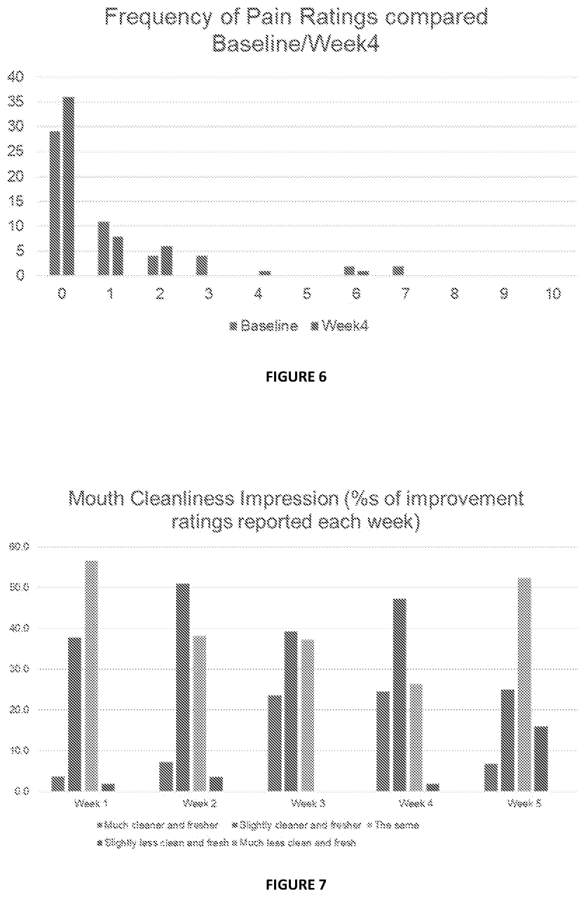

FIGS. 6, 7 and 8 show the effect on factors relating to canine periodontal disease after use of the protein biocomplex for 28 days;

FIG. 9 shows the effect on the perception of breath smell score after use of the protein biocomplex over 12 weeks; and

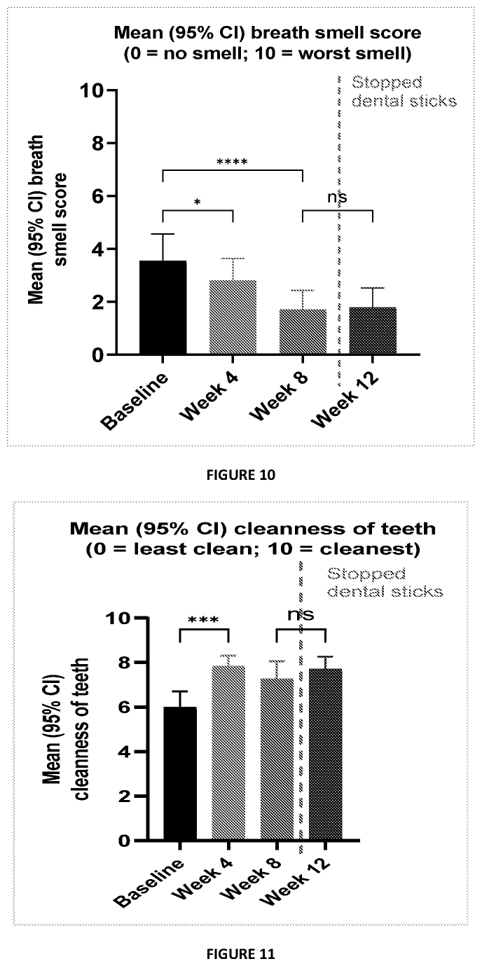

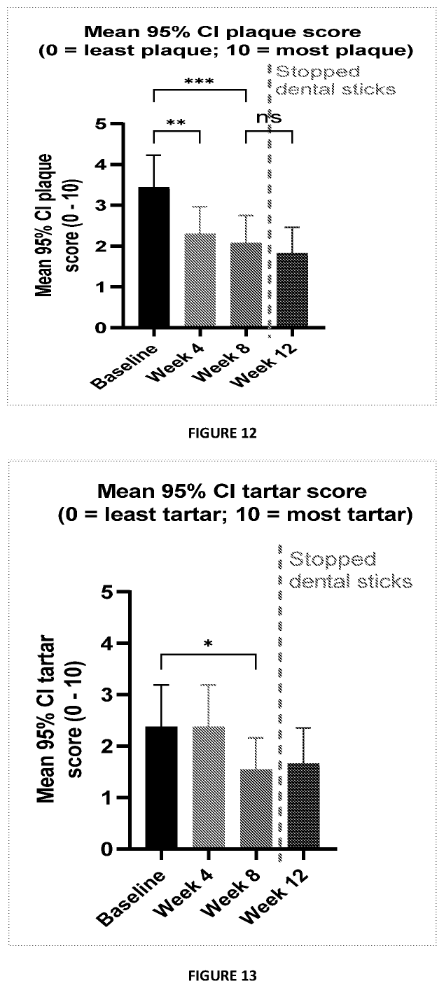

FIGS. 10, 11, 12 and 13 show the effect on factors relating to canine periodontal disease after use of the protein biocomplex according to an alternate dosage regimen.

### DETAILED DESCRIPTION

As noted above, described herein are methods and a complexed protein with useful effects in terms of stabilisation and, when applied topically, the ability to adhere the complexed proteins to an epithelial surface and retain excellent protein functionality.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refer to at least about 50%, for example 75%, 85%, 95% or 98%.

The term ‘comprise’ and grammatical variations thereof shall have an inclusive meaning—i.e. that it will be taken to mean an inclusion of not only the listed components it directly references, but also other non-specified components or elements.

For the purposes of this specification, the term ‘complex’, ‘bio-complex’ or grammatical variations thereof refers to a physical interaction between the microbiome regulating protein and the modified whey protein isolate (WPI) such that they bind to each other and, when topically applied, adhere to epithelial surfaces and, on adhesion, the microbiome regulating protein is functional. Without being bound by theory, it is understood by the inventor that the physical interactions retaining the complex together may be due to conjugation, physical entrapment or combinations thereof. Additional interactions may occur and, reference to conjugation or physical entrapment should not be seen as limiting.

The term ‘modified whey protein isolate’ or grammatical variations thereof as used herein refers to soluble native protein, protein aggregates and protein fibrils produced from whey protein isolates (WPIs).

For brevity of description, the word ‘protein’ or grammatical variations thereof as used herein is intended to encompass one protein, multiple proteins, a protein fraction, or protein fractions and reference to a singular protein should not be seen as limiting.

### Method of Producing a Complex

A method of producing a complexed stable microbiome regulating protein in accordance with the invention and configured for topical application is described herein, the method comprising the steps of: selecting a microbiome regulating protein in a powder form; selecting a modified whey protein isolate (WPI) from milk in a gel form; solubilising the microbiome regulating protein in an aqueous salt solution, the aqueous salt solution having an ionic strength of 25-200 mM NaCl; blending together the solubilised microbiome regulating protein and modified WPI; adjusting the pH to 2.0-6.0; optionally adjusting the temperature to 18-37° C.; holding the blend at a pH of 2.0-6.0 and a temperature of 18-37° C. for at least 30 minutes. It has been observed that the selective bactericidal activity of the protein described herein can be adversely effected if the temperature of any step of the process exceeds 37° C., and the pH drops below 2.0 or greater than 9.0.

The viscosity of the modified whey protein isolate is important to facilitate the complexing, and the optimum temperature for the most suitable viscosity is in the range of 18-37° C.

lonic strength of the mixture is important for the correct interaction of the protein groups on both components and the optimum ionic environment is maintained by 25-200 mM NaCl at pH 2.0-6.0.

Only through careful experimentation and balance of these parameters, were the useful effects were obtained. Indeed, the extent of adherence of protein on epithelial surfaces post complexing was more than double that of each component alone and in the inventors experience synergistic in nature or at least well beyond that anticipated suggesting a synergistic effect was possible but that could not have been previously expected or predicted.

### Complexed Form

The resulting complexed protein from the above method may be a semi-solid. The complexed protein may be a gel. The gel may have a viscosity equivalent to the modified WPI prior to complexing. The gel may have a viscosity of approximately 0.25 to 4.5 Poise.

The complexed protein may be stable and the complexed protein may be retained in complexed form by the modified WPI until applied topically at which point the protein function is made available to the epithelial surface that the modified WPI has adhered to.

### Microbiome Regulating Protein

The microbiome regulating protein may be a blend of protein fractions from milk that are selectively toxic to pathogenic bacteria, but which minimise any reduction in commensal bacteria population. The microbiome regulating protein may comprise: lactoperoxidase, lactoferrin, lysomal alpha-mannosidase, immunoglobulin G, angiogenin, ribonuclease 4, quiescin sulfhydryl oxidase, and combinations thereof. The protein may comprise all of the above listed proteins. The microbiome regulating protein selected for the above method may initially be in dried powder form.

### Modified WPI

The modified WPI selected for the above method may initially be in the form of an aqueous gel. The modified WPI selected may be derived from bovine species milk but may also be sourced from modified WPI obtained from other animals such as goats and sheep.

### Ratio

The ratio of microbiome regulating protein to modified WPI used in the method and/or present in the complexed protein may be: 1:1, or 1:2, or 1:3, or 1:4, or 1:5, or 1:6, or 1:7, or 1:8, or 1:9, or 1:10. In one embodiment, the ratio may be from 1:1 to 1:10; i.e. 1 part microbiome regulating protein to 1-10 parts modified whey protein isolate. In one embodiment, the ratio may be a 1:1 ratio. In the inventor's experience it may be necessary to have at least as much modified WPI to protein to ensure the desired extent of complexing is reached. Lower ratios could be used but with waste or non-complexed protein resulting from the method due to insufficient modified WPI being present at lower ratios.

### Salt Solution

The salt solution may comprise a mix of water and a chemical salt. In one embodiment, the chemical salt may be NaCl although other salts such as MgCl may also be used. Salt may be added to the extent needed to provide the indicated ionic strength. The ionic strength of the salt solution may be approximately 25, or 50, or 75, or 100, or 125, or 150, or 175, or 200 mM NaCl. As noted above, the ionic strength may vary from 25-200 mM NaCl. In one embodiment, the ionic strength be approximately 75 mM NaCl. This ionic strength remains/is maintained in the complexed blend of protein and modified WPI during and after holding. This ionic strength range in the inventor's experience appears to be an optimum for complexing or dissolution of the microbiome regulating protein into the modified WPI although, there may be some variation depending on the pH, temperature and holding time used. Use of a salt solution appears to be important as this creates the correct ionic nature of the solution to enable the powdered protein to dissolve and the protein to be correctly charged to create the complex.

### Blending

Blending occurs gently so that no or minimal foaming of the mixture occurs during blending. Blending may be completed, for example, using a planetary mixer. Blending may continue during pH adjustment, temperature adjustment and/or holding. Gentle or slow blending of the protein may be important to minimise or avoid denaturing and loss of functionality.

pH

The pH of the blend prior to pH adjustment may be approximately 6.5 to 7.5 or, around neutral pH 7.0. This start point in pH may be dependent on the pH of the water used to form the aqueous salt solution. The pH of the blend after pH adjustment may be approximately 2.0, or 2.5, or 3.0, or 3.5, or 4.0, or 4.5, or 5.0, or 5.5, or 6.0. In one embodiment as noted above, the pH may be from 2.0 to 6.0. In one embodiment, the pH may be approximately 4.0. This pH range and value appears to be an optimum for complexing or dissolution of the protein into the modified WPI although there may be some variation depending on the ionic strength, temperature and holding time used. The acid conditions post adjustment appear to maintain the appropriate charges on the protein to ensure bonding of the complex. The pH may be adjusted by use of an acid. The acid in one embodiment may be hydrochloric acid although other acids may also be used. In the inventor's experience, no buffers are required to maintain the reduced pH once adjusted.

### Temperature

The temperature of the blend after any adjustment (if needed) may be approximately 18, or 19, or 20, or 21, or 22, or 23, or 24, or 25, or 26, or 27, or 28, or 29, or 30, or 31, or 32, or 33, or 34, or 35, or 36, or 37° C. As noted above, the temperature post any adjustment may be from 18-37° C. In one embodiment, the temperature post adjustment may be approximately 25° C. The temperature may be adjusted to maintain the modified WPI at a desired viscosity to enable optimised complexing. This desired viscosity may be in the range of approximately 0.25 to 4.5 Poise. The temperature range and values described appear to be an optimum for complexing or dissolution of the proteins into the modified WPI although there may be some variation depending on the ionic strength, pH and holding time used. The temperature may be adjusted from ambient conditions (or retained at ambient conditions if the ambient temperature is within the desired range). Temperature adjustment may be up or down depending on the difference between the desired temperature and the ambient temperature.

### Holding Time

The holding time may be at least 30 minutes. The holding time may be from 30 minutes to 24 hours. In one embodiment, the holding time may be approximately 60 minutes. In the inventor's experience, most of the complexing occurs within 30-60 minutes based on the chosen parameters. Extended durations could be used and, based on the inventor's experience; there is no harm or risk of loss of functionality in holding the mixture for longer time periods. This timing appears to be an optimum for complexing or dissolution of the protein into the modified WPI although there may be some variation depending on the ionic strength, temperature and pH used.

### Drying

Optionally, drying may be completed in advance of subsequent formulation. In this embodiment, the dried complex may be a solid. Drying may be to a water activity of less than 0.6. In selected embodiments, the water activity may be as low as 0.2-0.3. Drying of the complex may be done under specific conditions of low temperature and low pressure over a time course that ensures the epithelial adhering nature and the selective bactericidal activity of the components of the complex are retained. Pressure during drying may be important in that, at low pressure the moisture boils off the gel at a lower temperature so that the drying can be undertaken at low temperatures. In addition, a very gradual lowering of pressure may be important so that the complexed protein solution in the dryer does not foam. Drying may be completed by freeze-drying or any other gentle drying process using a non-denaturing temperature/pressure e.g. less than 30° C., in a vacuum. The complexed protein remains functional when rehydrated post drying.

### Protein Complex of the Invention

There is provided a complexed protein or bio-complex produced by the method substantially as described above. Further, the invention provides a complexed protein comprising a microbiome functional protein complexed with a modified whey protein isolate (WPI) from milk, the complexed microbiome functional protein configured for topical application and, on application, to adhere to epithelial surfaces. The complexed protein may be a semi-solid aqueous gel with a viscosity of approximately 0.25 to 4.5 Poise. The complexed protein may be further formulated into an emulsion, gel, paste or putty for use within a dental composition. The microbiome regulating protein in the complex may be a blend of protein that are selectively toxic to pathogenic bacteria, but which minimise any reduction in commensal bacteria population. The protein in the complex may comprise: lactoperoxidase, lactoferrin, lysomal alpha-mannosidase, immunoglobulin G, angiogenin, ribonuclease 4, quiescin sulfhydryl oxidase, and combinations thereof. The modified WPI in the complex may be derived from bovine species milk.

The ratio of microbiome regulating protein to modified WPI in the complexed protein may be from 1:1 to 1:10. The complexed protein produced by themethod disclosed herein or with the features claimed has useful effects in terms of stabilisation. Further, when applied topically, the protein complex has the ability to adhere the functional proteins described to an epithelial surface and retain excellent protein functionality. This appears to be at least in part due to the important chemical and physical conditions under which the two components are reacted together to form the complex which may be controlled to optimise the functional activity of both components.

### Treatment Methods and Uses

Usefully, the complexed protein made herein is made suitable for the first time in use as a medicament, and particularly for use as a veterinary medicament. An increase in the pathogenic bacteria typically causes bad breath, plaque and gum disease in animals. The protein biocomplex can therefore be used to effectively treat an adverse health condition, such as an oral bacterial infection or a dental disease in an animal, where the condition or disease is due to an increase in pathogenic bacteria. The adverse health of the animal, cat or dog may also be due to a reduction in commensal bacteria, thereby creating an imbalance in the microbiota in the oral mucosa or specifically within the plaque. Such an imbalance may result in one or more conditions selected from bad breath (halitosis), plaque and/or gum disease and so the composition of the invention is useful in the treatment to restore that balance. In embodiments, the pathogenic bacteria may be psychrobacter. In particular, due to the selective functionlitiy that is able to be delivered for the first time in the oral mucosa, the complexed protein (or a composition comprising the same) is useful in the treatment of an oral or dental infection. In some embodiments, the infection may be gum or peridontal disease which has been mediated by an imbalance of pathogenic bacteria. Further, There is described herein a method of selectively treating an animal for a toxic pathogenic bacteria, but minimising any reduction in commensal bacteria population, by the step of topically administering a complexed protein, the complexed protein comprising a microbiome functional protein complexed with a modified whey protein isolate (WPI) from milk, the complexed protein configured for topical application; and on application, the complexed protein adheres to the animal epithelial surfaces. In the above method, the animal may be a non-human animal, optionally such as a cat or a dog. There is provided the use of a complexed protein comprising a microbiome functional protein complexed with a modified whey protein isolate (WPI) from milk, the complexed protein configured for topical application and, on application configured to adhere to epithelial surfaces, in the manufacture of a medicament for topical treatment of toxic pathogenic bacteria in or on an animal in need thereof, but minimising any reduction in commensal bacteria populations on epithelial surfaces of an animal.In the above use, the animal may be a non-human animal.

### Compositions Comprising the Protein Complex

In embodiments, the invention relates to a composition comprising the protein complex, which is made in accordance with the disclosure herein. Such a composition may comprise at least 0.5% to 2% w/w, preferably 0.1 to 1.5% w/w of the bio-complex and at least one or more pharmaceutically acceptable excipient. A composition comprising the protein complex is suitable to facilitate the selective antibacterial effect in the mouths of companion animals. This model has been shown to be effective in the described examples of the oral cavity of animals such as canines, in the examples herein, for the first time. An example of a suitable composition comprising the protein biocomplex in accordance with the embodiments above was tested in a suitable canine model trial to confirm the therapeutic effect. Provision of a suitable composition comprising the biocomplex, as claimed, has been shown to practically address specific oral challenges in dogs, such as dental disease and odour control.

Common excipients known in the art may be used to formulate such a composition; these include bulking agents such as rice, oat flour, sweet potato flour and/or other plant-based flour. These may be selected alone with a combination of other well-known excipients useful in veterinary food supplements or dental compositions. Such excipients may further include humectant, optionally at 15-20% w/w, flavouring, optionally at 4-6% w/w, pH buffer, optionally at 3-5% w/w, lubricant, optionally at 1-3% w/w, emulsifier, optionally at 1-2% w/w, thickener, optionally at 0.5-1% w/w and one or more preservatives, optionally of up to 0.01% w/w.

To this end a composition comprising the protein biocomplex can be used to effectively treat an adverse health condition, such as an oral bacterial infection or a dental disease in an animal, particularly where the condition or disease may be due to an increase in pathogenic bacteria. An increase in the pathogenic bacteria typically causes bad breath, plaque and gum disease in animals. The adverse health of the animal, cat or dog may be due to a reduction in commensal bacteria, thereby creating an imbalance in the microbiota in the oral mucosa or specifically within the plaque. Such an imbalance may result in one or more conditions selected from bad breath (halitosis), plaque and/or gum disease and so the composition of the invention is useful in the treatment to restore that balance. In embodiments, the pathogenic bacteria may be psychrobacter. Such a composition may also enable a temporary physical barrier configured to deliver the protein biocomplex more effectively and provide the biocomplexed protein and its anti-bacterial character at the required site (on the gums and teeth of an animal), whilst working with the oral microbiome to support the commensal flora an animal requires for maintaining good oral health. In embodiments, the animal is a cat or a dog. In embodiments, the disease may be periodontal disease.

The composition should be formulated to be gentle on the digestive system of the animal in order to be employed for frequent use and/or part of a long-term viable solution to maintain oral health of the animal over a reasonable period of months or years.

In embodiments, when the composition is used for the treatment and/or improvement of oral health and more specifically, dental health, a suitable dosage regimen can be followed. To reduce the pathogenic numbers and improve oral health significantly such a composition has been shown to be effective when used including the required minimum amount (therapeutically effective) of biocomplex-be administered daily for at least 5 days per week, preferably for at least 4 weeks, 5-12 weeks or longer. Thereafter, a reduced frequency of dosing every other day may be utilised to maintain health in a suitably re-balanced microbiome of the oral mucosa. However, regular administration will more effectively prevent re-occurrence of pathogen build-up and therefore maintain a suitably re-balanced oral mucosa.

Without being bound by theory, such a composition may include further pharmaceutical excipients to enhance the protective function of the composition whilst the product is delivered and then held in the mouth of the subject. Such a dental composition could provide for a more effective and prolonged but precise release of the bioactive in the appropriate location to achieve a desired efficacy. However, it is plausible it would be relevant to other animals especially those with an elongate jaw particularly, such as cats.

In embodiments, the biocomplexed protein be distributed evenly throughout the composition, or the composition may form an outer structure, which fully surrounds the protein element. It is understood by the inventor that the complexed protein produced binds to other epithelial surfaces and may be used to maintain or re-establish a healthy microbiome on any epithelial surface e.g. skin and nasal cavities, the gut and so on. This could include products for humans and animals, skin care products for human and animals, nasal spray products for humans and animals, and ingestible gut health products for humans and animals. It is also envisaged that the complexed protein may be used to maintain, establish or re-establish a healthy microbiome on any protein-rich surface such as: Hair, including hair products for humans; lotions, creams, shampoos, conditioners, sprays. Animal coats-lotions, creams, shampoos, conditioners, sprays. Medical devices including artificial skin, wound repair collagen scaffolding, bandages etc. As should be appreciated form the above, topical surfaces may be external e.g. the skin, or internal e.g. the gut. Reference to the term ‘topical’ in this specification is not limited to only external application of the complexed protein.

The embodiments described above may also be said broadly to consist in the parts, elements and features referred to or indicated in the specification of the application, individually or collectively, and any or all combinations of any two or more said parts, elements or features.

The above methods, protein biocomplex, compositions and uses and applications thereof are exemplified in further detail below.

### Example 1—An Outline is Provided of the Method of Production of the Complex:

### Example 2

Adherence of the Complex to Epithelial Surfaces

To determine if the complex was adherent to epithelial layers a portion of pig's skin was soaked in a solution of 50 mg/ml of the complex (containing 50 mg/mL protein), washed twice in phosphate buffer at pH 7.4 and stained with Coomassie Blue, which stains for protein. This gave an intensely stained skin compared to pig's skin that had not been soaked in the complex (FIG. 1). Treatment of pig skin with 50 mg/ml modified whey protein isolate fraction (containing 50 mg/mL protein) or the microbiome regulating proteins from bovine milk (containing 50 mg/ml protein) alone, then washed, showed some binding of these components to the skin, but not as much as with 50 mg/ml of the complex of both components (Table 1).

TABLE 1
Portion of pig skin treated without or with the complex, and
the components of the complex, washed twice and stained for
protein attached to the epidermal layer of the pig's skin.

| | | Pig skin soaked | | |
|---|---|---|---|---|
| | | in 50 mg/ml of | | |
| | | microbiome | Pig skin soaked | |
| | | regulating | in 50 mg/ml | Pig skin soaked in |
| | | proteins from | modified whey | 50 mg/ml of the |
| | | bovine milk for | protein isolate | complex for 60 |
| | | 60 secs, then | for 60 secs | secs then |
| Treatment | Pig skin | washed. | then washed. | washed. |
| Relative | 1.0 | 1.13 | 1.63 | 2.35 |
| increase in | | | | |
| protein | | | | |
| binding | | | | |

The epithelial binding activity of the complex was much greater (more than double) than the modified whey protein isolate alone or the microbiome regulating proteins alone. The modified whey protein isolate has been specifically prepared to attach to epithelial surfaces. Thus, it would be expected that the amount of binding to the pig's skin of this component would be similar to the amount of binding of the complex. However, the complex binding was 44% greater than the modified whey protein isolate alone, suggesting a synergistic effect of the components, or that the process used to generate the complex was increasing the adhering ability of the complex.

### Example 3

### Retention of the Enzyme Bioactivity of the Complex

The enzyme lactoperoxidase is a protein that has been implicated in the microbiome regulating proteins isolated from bovine milk (NZ547859 and PCT/NZ2017/050043). Lactoperoxidase activity was measured by the addition of the complex, or the components of the complex separately, to a solution of hydrogen peroxide in 100 mM Phosphate buffer, pH 5.5, and monitoring the oxidation of 2,2-Azino-bis(3-Ethylbenzthiazoline-6-sulphonic acid) (ABTS) spectrophotometrically at 436 nm.

Lactoperoxidase activity of the combination of the microbiome regulating proteins from bovine milk was retained when the microbiome regulating proteins were complexed with the modified whey protein isolate (Table 2). As would be expected the modified whey protein isolate had no peroxidase activity.

TABLE 2
Lactoperoxidase activity of the complex compared
to the components of the complex.

| | Lactoperoxidase |
|---|---|
| Component | activity (Units) |
| Modified whey protein isolate (50 mg/ml) | 0 |
| Microbiome regulating proteins (including | 205005 |
| lactoperoxidase 16 mg/ml) | |
| Complex of whey protein isolate and protein | 223339 |
| fractions from bovine milk (50 mg/ml) | |

### Specific Bactericidal Activity

### Commensal Bacteria

Microbiome regulating proteins isolated from bovine milk have been shown to have no effect on the growth of commensal bacteria such as those from the Lactobacillus family PCT/NZ2017/050043. In order to determine the effect of the complex containing these microbiome regulating proteins on a commensal bacteria Lactobacillus acidophilus bacteria were cultured anaerobically and the complex was added to the cultures and numbers of bacteria after 24 hours were determined as the optical density at 650 nm. The complex of the modified whey proteins and the microbiome regulating protein isolated from bovine milk had no effect on the growth of the commensal bacteria Lactobacillus. acidophilus (FIG. 1)

### Pathogenic Bacteria

Microbiome regulating proteins isolated from bovine milk have been shown to be bactericidal to pathogenic bacteria such as E. coli PCT/NZ2017/050043. In order to determine the effect of the complex containing these protein on E. coli these bacteria were cultured and the complex was added to the cultures and numbers of bacteria after 24 hours were determined as the optical density at 650 nm. The complex inhibited growth of the pathogenic bacteria E. coli to a similar extent as the microbiome regulating protein from bovine milk (FIG. 2). Note that the square data points in FIG. 2 correspond to results seen for addition of the microbiome regulating proteins from bovine milk and the circular data points correspond to results seen for addition of the complex of the microbiome regulating proteins with the modified whey protein isolate.

### Effect of the Complex Bound to Skin on Growth of Pathogenic Bacteria

S. aureus is a pathogenic bacteria often associated with skin disease. The microbiome regulating proteins isolated from bovine milk has been shown to be bactericidal to S. aureus grown in culture PCT/NZ2017/050043. Samples of pig skin treated with the microbiome regulating proteins from bovine milk then washed showed a modest inhibition of S. aureus growth. However, samples of pig skin treated with the complex, then washed showed significantly greater inhibition of S. aureus growth (FIG. 3). This confirms the results seen in Table 1 confirming that the complex binds well to epithelial surfaces such as skin, and also retains the bactericidal activity of the microbiome regulating proteins from cow's milk.

### Example 4

A formulated composition should comprise a minimum therapeutically effective amount of the protein biocomplex. In a formulated product this might yield a final product of a sufficient total size, for example that a dog could hold in its mouth. In examples this may include approximately 0.05%-2% w/w of the protein biocomplex in the composition, preferably 0.13% to 0.53% w/w.

In this example, a composition to be tested on the animals (dogs) was formulated for lighter or toy dog breeds (up to 15 kg), medium weight breeds (15-31 kg) and bigger, heavier breeds (31 kg+). The protein biocomplex in the given composition example included 0.13% w/w (where a larger sized composition was needed for the heavy breeds) or 0.5% w/w (where a smaller sized composition is needed) as necessary.

Such a composition may be made by blending (in accordance with well-established techniques) the protein bio-complex component with other biologically acceptable excipient components known for use in veterinary food supplement products. For example, the known components and suitable ranges provided in the table below may exemplify components utilised in embodiments of the composition comprising the protein biocomplex.

| | |
|---|---|
| Emulsifier | 5-10% |
| Water | 5-10% |
| Edible oil(s) | 5-15% |
| Humectant | 20-30% |
| Bulking agent(s) | 35-40% |
| Preservative | 0.1-0.5% |
| Flavouring | 3-5% |
| Thickener | 2-5% |

### Selective Bactericidal Activity

A composition according to the above-referenced Example 4 was incubated (in accordance with well-known methods in the art) with cultures of both pathogenic and commensal bacteria isolated from a single canine oral source to determine if it retained its selective bactericidal activity.

The result confirmed that the number of canine pathogenic oral bacteria from 108 colony-forming units (cfu) to 107 cfu after 18 hours of incubation compared to a control.

There was no reduction in the number of canine commensal oral bacteria after 18 hours incubation. This confirmed that the selective bactericidal activity required for application in animals is retained within an example composition of the protein biocomplex.

This composition (as per Example 4) was then utilised in the following canine studies:
Preliminary Canine Studies—Short Term (1 to 4 Weeks)

A preliminary microbiome study was conducted to assess the therapeutic effect of an example composition comprising the protein biocomplex and thus its suitability for veterinary use.

Eleven (11) dogs were each given a composition (in accordance with the invention embodied in Example 4) every day for five (5) consecutive days (day 2, day 3, day 4, day 5 and day 6 of the test regimen). Before and after swabs of the oral and faecal microbiome were taken for each dog and a final oral swab 48 hours. At the end of the trial, the oral and faecal swabs were collected and DNA was successfully extracted for analysis in appropriate lab conditions.

A full microbiome profile analysis was undertaken using 16s Amplicon Sequencing and Analysis (Amplicon Normalization, PCR reaction, Amplicon Barcoding, Amplicon Library QC, Paired end reads, 2×250 bp read length). Deep amplicon sequencing, combined with the principles of statistical ecology were used to survey microbiome communities. Deep sequencing of the small ribosomal rRNA subunit (16S rRNA) gene was used to survey the bacterial community and gain insights on the population dynamics, specifically the oral pathogenic bacteria psychrobacter. The result for the psychrobacter levels in oral samples is shown by a graphic analysis in FIG. 4. This plot illustrates the mean and standard error of the relative abundances of psychrobacter.

There are 3 adjacent time bars shown for psychrobacter in the oral swab taken in accordance with the study protocol:
The first is at Time 1=before the composition comprising protein biocomplex was given (day 2)
The second is Time 2=after 5 days (day 6)
The third bar is Time 3=48 hours after the last composition given (day 8)

It can be seen on the far right of FIG. 4 that psychrobacter (a proteobacteria), which is known to cause dental disease, such as periodontal disease is significantly reduced after 5 days consecutive application to the dogs. The levels of this bacterium remain low, despite no further application, 2 days later. The results herein therefore conclusively demonstrate that a composition comprising the protein biocomplex in a therapeutically effective amount reduces pathogenic bacteria that are specifically present in the plaque and thus enables a targeted and effective treatment (or prevention) of dental disease that is associated therewith. Initially, it may be that a suitable dosage regimen to reduce the pathogenic bacteria would be one application daily for at least 5 days. This may be followed immediately by a regimen with a reduced frequency of every other day, since it is likely that regular continuous administration is required to prevent re-occurrence of high levels of pathogen and therefore specifically maintain dental health, once the bacterial flora are suitably re-balanced.

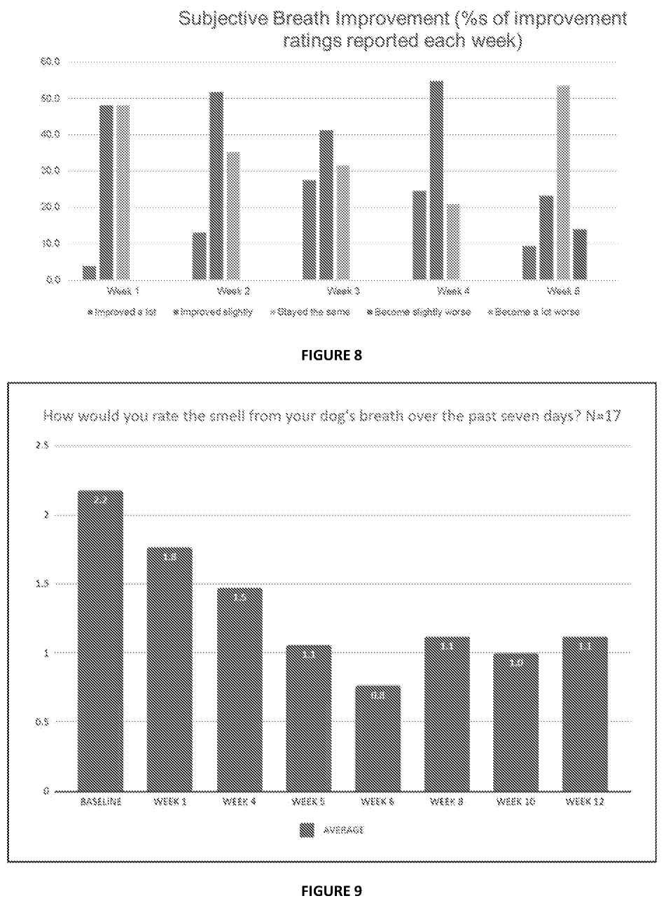

Further, since psychrobacter is associated and likely responsible for malodour in (bloodhound) dogs a reduction in the adverse and unpleasant symptoms of disease such as malodour of breath is plausible. Further, in accordance with the study, the faecal samples obtained before and after the 5 day trial, were also sent for microbiome profiling and analysis (as detailed above). Importantly, as can be seen from FIG. 5, this showed no significant acute effect on the stool microbiome in response to 5 days of use, suggesting the product is suitable for selectively targeting disease-causing bacteria in the mouth without effecting the microbiome of the gut. Following this, an extended version of the same trial over 28 days (4 weeks) was found to confirm these findings, indicating a statistically relevant:
reduction in oral pain linked to issues such as gum infections, tooth root infections, loose teeth after week 4 (see FIG. 6)
reduction in presence of tartar and plaque likely improved mouth cleanliness (see FIG. 7) and breadth (see FIG. 8)—improvement by week 3 and 4 (and decline in week 5 after composition was not used for 1 week)
   Canine Study 2—Medium Term (12 weeks)

A 12 week dental trial was conducted to further evaluate the effect of the protein biocomplex as it related to factors linked with periodontal disease in dogs.

A total of 17 dogs aged>6 months, with persistent dental issues such as gum disease, tartar, plaque, bad breath and a weight between 15->30 kgs were engaged in the trial. Dogs with healthy teeth were excluded.

Regimen: the composition (as previous disclosed) was used every day for 4 weeks, followed by every other day for 8 weeks.

### Factors Assessed and Results

Assessments were made at Baseline (Week 0), Week 1, Week 4, Week 5, Week 6, Week 8, Week 12. It was established that a significant improvement was reported in dog's breath within 4 weeks and breath freshness was maintained with continued use over the full 12 weeks, even with reduced frequency of administration to alternate days. It was observed that longer-term use (around 8 weeks) was required to see a more significant impact and improvement on other factors such gum bleeding, plaque and tartar.

### Breath Smell and Cleanliness

As can be seen in FIG. 9, a gradual reduction in the perception of breath smell score was reported with a decline in the average breath score from 2.2 in baseline to 1.5 by week 4 after daily use. Additional use (4 weeks +) showed a gradual decrease in breath score even after a change of frequency (every other day). For example, the lowest average breath score was 0.8 reported in week 6. Overall, a lower average breath score, less than half, than in baseline (2.2) was reported in week 5, 8, 10 and 11 (between 1.0-1.1) towards the end of the trial.

The proportion of responses for “Slightly more clean and fresh” and “Much more clean and fresh” was higher in weeks 4-12 compared to week 1. After just 4 weeks, more than 75% of owners reported that their dog's mouth was (slightly or much) more clean and fresh.

### Gum Bleeding

Of those owners who brush their dog's teeth, there was a decline in the proportion of those whose gum bled from 11.8 at baseline to 5.9% by week 8.

### Plaque

By week 8 a reduction in plaque was evident: owners reporting plaque score from 76.5% at baseline to 29.4%

### Canine Study 3-Alternate Dosing Regimen (12 Weeks)

A further 12-week trial was completed to assess the efficacy of the protein biocomplex (as previously formulated) when administered every other day. In this trial, a larger sample size of dogs was involved: 35 dogs began the trial including, 13 small breeds (up to 15 kg), 11 medium breeds (15-31 kg) and 11 large breeds (over 31 kg). This was important to assess and confirm the consistency of the effect for different weight categories of dog.

### Trial Process

Each dog was given the protein biocomplex composition of a size suitable for their weight category (in accordance the examples described in Example 4) every other day over a period of 10 weeks.

### Factors Assessed and Results:

An assessment survey was taken at baseline, 4 weeks, 8 weeks and at 12 weeks (2 weeks after no administration). The microbiome and health of the dog was then assessed by a number of criteria by their owners:

As can be seen in FIGS. 10, 11 and 12, after 4 weeks, there was a significant improvement in breath smell score, cleanness of teeth and plaque score. As can be seen in FIG. 13, after 8 weeks, there was a significant improvement in tartar score.

All changes seen by week 8 were maintained to week 12 but there were no significant changes noted between week 8 and week 12.

Statistical relevance—participants missing survey responses after baseline were included in the analysis. For each question, a mixed effects model was used to analyse significant differences between mean scores for each survey during the trial. Post-hoc multiple comparison used Tukey's test to compare the mean score at baseline with week 4 and week 8 and at week 8 with week 12. (to account for missing values). Significant differences were defined by p<0.05.

1. A complexed protein comprising:
a microbiome functional protein comprising lactoperoxidase, lactoferrin, lysomal alpha-mannosidase, immunoglobulin G, angiogenin, ribonuclease 4 or quiescin sulfhydryl oxidase and/or combinations thereof, complexed with a modified whey protein isolate (WPI) from milk, wherein the complexed microbiome functional protein is configured for topical application and, on application, to adhere to epithelial surfaces.

2. The complexed protein as claimed in claim 1, wherein the complexed protein is a semi-solid aqueous gel with a viscosity of approximately 0.25 to 4.5 Poise.

3. The complex protein as claimed in claim 1, wherein the microbiome functional protein is a blend of protein fractions that are selectively toxic to pathogenic bacteria, but which minimise any reduction in commensal bacteria population.

4. The complexed protein of claim 1, wherein the modified WPI is derived from bovine species milk.

5. The complexed protein of claim 1, wherein the ratio of microbiome regulating protein to modified WPI in the complexed protein is from 1:1 to 1:10.

6. The complexed protein of claim 1, in therapeutically effect amount for use as a medicament.

7. (canceled)

8. Use of the complexed protein of claim 1, in the manufacture of a medicament for topical treatment of toxic pathogenic bacteria, or on an animal in need thereof, but minimising any reduction in commensal bacteria populations on epithelial surfaces of an animal.

9. A method of selectively treating an animal for a toxic pathogenic bacteria, but minimising any reduction in commensal bacteria population, by the step of: topically administering a complexed protein as claimed in claim 1 and on application, the complexed protein adheres to the animal epithelial surfaces.

10. The use or method as claimed in claim 8, wherein the animal is a non-human animal.

11. A topical composition comprising the complexed protein of claim 1 and further comprising at least one pharmaceutically acceptable excipient.

12. A method of producing a complexed stable microbiome regulating protein configured for topical application, the method comprising the steps of:
selecting a microbiome regulating protein wherein the protein comprises:
lactoperoxidase, lactoferrin, lysomal alpha-mannosidase, immunoglobulin G, angiogenin, ribonuclease 4, or quiescin sulfhydryl oxidase, and/or combinations thereof in a powder form;
selecting a modified whey protein isolate (WPI) from milk in a gel form;
solubilising the microbiome regulating protein in an aqueous salt solution, the aqueous salt solution having an ionic strength of 25-200 mM NaCl;
blending together the solubilised microbiome regulating protein and modified WPI;
adjusting the pH to 2.0-6.0;
adjusting the temperature to 18-37° C.; and
holding the blend at a pH of 2.0-6.0 and a temperature of 18-37° C. for at least 30 minutes.

13. The method as claimed in claim 12, wherein the microbiome regulating protein is a blend of protein fractions that are selectively toxic to pathogenic bacteria, but which minimises any reduction in commensal bacteria population.

14. The method as claimed in claim 12, wherein the complexed protein produced is a semi-solid gel with a viscosity of approximately 0.25 to 4.5 Poise.

15. The method of claim 12 to, wherein the modified WPI selected is initially in the form of an aqueous gel.

16. The method of any of claim 12, wherein the modified WPI selected is derived from bovine species milk.

17. The method of claim 12, to 16, wherein the ratio of microbiome regulating protein to modified WPI used in the method and present in the complexed protein is from 1:1 to 1:10.

18. The method of claim 12, wherein the salt solution comprises a mix of water and a chemical salt.

19. The method of claim 12, wherein the ionic strength of the salt solution is approximately 75 mM NaCl.

20. The method of claim 12, wherein the pH of the blend prior to pH adjustment is approximately 6.5 to 7.5.

21. The method of claim 12, wherein the pH of the blend after pH adjustment is approximately 4.0.

22. The method of claim 12, wherein the temperature of the blend after any adjustment is approximately 25° C.

23. The method of claim 12, wherein the holding time is approximately 60 minutes.

24. The method of claim 12, wherein the complexed protein is dried after holding to a water activity of less than 0.6.

25. A complexed protein produced by the method as claimed in claim 12.
